(19) United States
(12) Patent Application Publication
(10) Pub. No.: US 2012/0171660 A1
(43) Pub. Date: Jul. 5, 2012

(54) METHOD FOR SCREENING AND PURIFYING ENTEROVIRUS, METHOD FOR MASS-PRODUCING ENTEROVIRUS, AND METHOD FOR MANUFACTURING ENTEROVIRUS VACCINE

(75) Inventors: Huan-Yao LEI, Tainan City (TW); Chia-Ming Liu, New Taipei City (TW)

(73) Assignee: National Cheng Kung University, Tainan City (TW)

(21) Appl. No.: 13/182,702

(22) Filed: Jul. 14, 2011

(30) Foreign Application Priority Data
Jan. 5, 2011 (TW) 100100378

(51) Int. Cl. C12N 7/02; C12N 7/04; C12Q 1/70

(52) U.S. Cl. 435/5; 435/239; 435/236

(57) ABSTRACT

The present invention relates to methods for screening or purifying enteroviruses, a method for mass-producing enteroviruses, and a method for manufacturing an enterovirus vaccine. The method for screening enteroviruses in a sample comprises the following steps: (A) providing a sample and a carrier, wherein monosaccharides such as glucose or galactose are bound to the surface of the carrier, and the monosaccharides have binding affinity to enterovirus; (B) contacting the sample with the carrier; (C) removing components of the sample that do not bind to the carrier; (D) providing a detection unit and contacting the detection unit with the carrier, wherein the detection unit binds to the sample bound on the carrier; and (E) measuring a signal of the detection unit, wherein when the signal of the detection unit is detected, it represents that the enterovirus exists in the sample.

### CROSS REFERENCE TO RELATED APPLICATION

This application claims the benefits of the Taiwan Patent Application Serial Number 100100378, filed on Jan. 5, 2011, the subject matter of which is incorporated herein by reference.

### BACKGROUND OF THE INVENTION

1. Field of the Invention

The present invention relates to methods for screening and purifying an enterovirus, a method for mass-producing an enterovirus, and a method for manufacturing an enterovirus vaccine and more particularly, to methods for screening and purifying an enterovirus, a method for mass-producing an enterovirus, and a method for manufacturing an enterovirus vaccine by use of monosaccharides.

2. Description of Related Art

Enteroviruses are a genus of +ssRNA virus belonging to the family of Picornaviridae. Among all types of enteroviruses, Enterovirus 71 (EV71) especially causes severe symptoms. Enterovirus 71 is a single stranded RNA virus, which is notable as one of the major causative agents for hand-foot and mouth disease (HFMD) or Herpangina. Sometimes, EV71 may further cause severe central nervous system diseases, which include: brainstem encephalitis, encephalitis, meningoencephalitis, aseptic meningitis, or acute flaccid paralysus (AFP). Among these central nervous system diseases, brainstem encephalitis may be complicated by pulmonary oedema and heart failure, and cause deaths.

EV71 was first isolated in 1969, widespread around the world. In addition, EV71 also causes severe encephalitis and polio-like syndrome. In 1998, EV71 caused a large outbreak in Taiwan, and the complications of neurogenic shock and pulmonary oedema caused the death of 78 children due to EV71 infection. Hence, EV71 is considered as an important neurotropic virus after poliomyelitis virus.

The central nervous system diseases caused by EV71 are quite severe. If the infection of EV71 in children can be detected in the early stage to perform a suitable treatment, the cure rate of EV71 can be greatly improved and the death rate thereof can further be greatly reduced. Hence, it is desirable to develop a method for screening a sample for the presence of an enterovirus, which can be used to screen the infection of enteroviruses in a simple and quick way, in order to perform a proper treatment in the early stage.

In addition, vaccines against enteroviruses also can be used to reduce the risk of the infection of enterovirus. Currently, many countries and companies are focused on the development of vaccines against enteroviruses. The commercial formulations of the vaccines against enteroviruses comprise: DNA vaccines, subunit vaccines, virus-like particle vaccines, and whole virus vaccines. Herein, the efficacy of the whole virus vaccines is most notable. However, when whole virus vaccines are produced, a large amount of enteroviruses must be cultured and purified in order to mass-produce vaccines for inoculation against enteroviruses. Hence, it is also desirable to develop methods for mass-producing and purifying enteroviruses, in order to obtain a large amount of enteroviruses suitable for vaccine production.

### SUMMARY OF THE INVENTION

The object of the present invention is to provide a method for screening enteroviruses, in order to simply and quickly detect whether enteroviruses exist in a sample or not.

Another object of the present invention is to provide a method for purifying enteroviruses, in order to simply and quickly obtain a large amount of enteroviruses.

A further object of the present invention is to provide a method for mass-producing enteroviruses, which can be used to obtain a large amount of enteroviruses for enterovirus-related research or the development of vaccines against enteroviruses.

A further other object of the present invention is to provide a method for manufacturing an enterovirus vaccine, in order to large scale manufacture enterovirus vaccines with complete viral particles.

To achieve the object, the method for screening a sample for the presence of an enterovirus of the present invention comprises the following steps: (A) providing a sample, and a carrier, wherein monosaccharides are bound to a surface of the carrier, and the monosaccharides have a binding affinity to the enterovirus; (B) contacting the sample with the carrier; (C) removing components of the same that do not bind to the monosaccharides on the carrier; (D) providing a detection unit, and contacting the detection unit with the carrier, wherein the detection unit binds to the sample bound to the monosaccharides on the carrier; and (E) measuring a signal of the detection unit, wherein when the signal of the detection unit is detected, it represents that the enterovirus exists in the sample.

The method for screening a sample for the presence of an enterovirus of the present invention is performed, based on the specific binding between the enteroviruses and the monosaccharides. When this method is applied for enterovirus detection, it is possible to screen in a simple and quick way whether enteroviruses exist in the sample or not. In addition, the monosaccharides used in this method of the present invention are easily available and inexpensive, so the cost of screening for enterovirus presence in the sample can be further reduced.

According to the method for screening a sample for the presence of an enterovirus of the present invention, the monosaccharides can be directly bound to the surface of the carrier; or the monosaccharides are bound to the surface of the carrier through lectins, in the step (A). Furthermore, the detection unit used in this method may comprise an anti-enterovirus antibody, or a monosaccharide connecting with a fluorescence dye or a phosphorescence dye. Preferably, the detection unit used in this method comprises an anti-enterovirus antibody. More preferably, the detection unit used in this method further comprises a horseradish peroxidase-conjugated antibody, which is an enzyme generally used in enzyme-linked immunosorbent assay (ELISA) and connects to the anti-enterovirus antibody. When the anti-enterovirus antibody is used as the detection unit, the specific binding between the anti-enterovirus antibody and the enterovirus can increase the accuracy of this method.

In addition, the present invention further provides a method for purifying an enterovirus, which comprises the following steps: (A) providing carriers, wherein monosaccharides are bound to surfaces of the carriers; (B) mixing an enterovirus-containing solution with the carriers, wherein enteroviruses contained in the enterovirus-containing solution bind to the monosaccharides on the carriers; (C) washing the carriers to remove components contained in the enterovirus-containing solution which are not bound to the carriers; and (D) providing a monosaccharide solution to separate the enteroviruses from the monosaccharides on the carrier.

The method for purifying the enterovirus of the present invention is achieved by the specific binding between the enteroviruses and the monosaccharides. When the enterovirus-containing solution is mixed with the carriers, the enteroviruses contained in the enterovirus-containing solution can bind to the monosaccharides on the carrier. Then, the enteroviruses bound to the monosaccharides are separated from the carriers through the competition reaction between the highly concentrated monosaccharide solution and the monosaccharides on the carriers. According to the method for purifying the enterovirus of the present invention, the enteroviruses can be quickly purified from the enterovirus-containing solution by the use of monosaccharides, which are easily available and inexpensive.

According to the method for purifying the enterovirus of the present invention, the monosaccharides can be directly bound to the surface of the carrier; or the monosaccharides can be bound to the surface of the carrier through lectins, in the step (A).

Furthermore, the present invention provides a method for mass-producing an enterovirus, which comprises the following steps: (A) providing host cells and an enteroviruses; (B) mixing the host cells and the enteroviruses in a monosaccharide-containing medium to transfect the enteroviruses into the host cells; (C) incubating the host cells transfected with the enteroviruses; and (D) extracting the enteroviruses from the host cells.

According to the method for mass-producing an enterovirus of the present invention, monosaccharides are added into the medium during a stage of virus absorption onto the host cells (i.e. the step (B)). The monosaccharides can facilitate the viruses being absorbed onto the host cells, and the replication of the viruses, to thereby increase the productivity of the enteroviruses. Hence, a large amount of the enteroviruses can be produced by the use of this method, and the obtained enteroviruses can be applied to enterovirus-related research or the development of vaccines against enteroviruses.

According to the method for mass-producing an enterovirus of the present invention, the host cells transfected with the enteroviruses can be incubated in a monosaccharide-containing medium, in the step (C). The monosaccharides may not only facilitate the enterovirus absorption (i.e. the step (B)), but also increase the replication of the enteroviruses after virus infection (i.e. the step (C)). In addition, the content of the monosaccharides in the monosaccharide-containing medium can be 0.03-1.0 M.

Furthermore, according to the method for mass-producing an enterovirus of the present invention, the enteroviruses in the host cells can be taken out by lysing the host cells to obtain an enterovirus-containing solution, and then the method for purifying an enterovirus of the present invention can further be used to extract the enteroviruses in the enterovirus-containing solution (i.e. the step (D)). Therefore, the method for mass-producing an enterovirus of the present invention may further comprise the following steps: (D1) providing carriers, wherein monosaccharides are bound on surfaces of the carriers; (D2) lysing the host cells to obtain an enterovirus-containing solution; (D3) mixing the enterovirus-containing solution with the carriers, wherein enteroviruses contained in the enterovirus-containing solution bind to the monosaccharides on the carriers; (D4) washing the carriers to remove components contained in the enterovirus-containing solution which are not bound to the carriers; and (D5) providing a monosaccharide solution to separate the enteroviruses from the monosaccharides on the carrier. In addition, the monosaccharides can be directly bound to the surface of the carrier; or the monosaccharides can be bound to the surface of the carrier through lectins, in the step (D1).

The present invention further provides a method for manufacturing an enterovirus vaccine, which comprises the following steps: (A) providing host cells and enteroviruses; (B) mixing the host cells and the enteroviruses in a monosaccharide-containing medium to transfect the enteroviruses into the host cells; (C) incubating the host cells transfected with the enteroviruses; (D) extracting the enteroviruses from the host cells; and (E) deactivating the enteroviruses extracted from the host cells.

The method for manufacturing an enterovirus vaccine of the present invention comprises: the steps of the methods for mass-producing an enterovirus and purifying an enterovirus (i.e. the steps (A)-(D) of the method for manufacturing an enterovirus vaccine); and a step of deactivating the enteroviruses. Therefore, the vaccine against enteroviruses can be quickly mass-produced by use of the methods of the present invention.

In addition, according to the method for manufacturing an enterovirus vaccine of the present invention, the enteroviruses extracted from the host cells can be deactivated by conventional deactivating methods generally used in the art. For example, the enteroviruses extracted from the host cells can be deactivated with formaldehyde.

According to the aforementioned methods of the present invention, the enterovirus can be Enterovirus species A virus. Preferably, the enterovirus is Enterovirus 71 (EV71), or Coxsackievirus A16 (Cox A16, CA16). More preferably, the enterovirus is Enterovirus 71. In addition, according to the aforementioned methods of the present invention, the monosaccharides can be glucoses, galactoses, or N-acetyl galactosamines. Preferably, the monosaccharides are glucoses. In addition, according to the aforementioned methods of the present invention, the lectins can be galectin-1, Concanavalin A (Con A), Lens culinaris agglutinin (LCA), Wheat germ agglutinin (WGA), Dolichos biflorus (DBA), or Ricinus lectin (RCA). Preferably, the lectins are galectin-1.

Other objects, advantages, and novel features of the invention will become more apparent from the following detailed description when taken in conjunction with the accompanying drawings.

### BRIEF DESCRIPTION OF THE DRAWINGS

FIGS. 1A-1C are graphs of binding assays according to Embodiment 1 of the present invention, which show EV71 binds to various kinds of monosaccharides, wherein “*” represents p<0.05 on T-TEST;

FIGS. 1D-1F are graphs of binding assays according to Embodiment 2 of the present invention, which show EV71 binds to various kinds of monosaccharides, wherein “*” represents p<0.05 on T-TEST;

FIGS. 2A-2E are graphs of binding assays according to Embodiment 3 of the present invention, which show EV71 binds to various kinds of lectins, wherein “*” represents p<0.05 on T-TEST;

FIG. 3 is graphs of binding assays according to Embodiment 4 of the present invention, which show EV71 binds to various kinds of lectins, wherein “*” represents p<0.05 on T-TEST;

FIGS. 4A-4C are graphs of assays showing the influence of monosaccharides on the replication of EV71 according to Embodiment 5 of the present invention, wherein “*” represents p<0.05 on T-TEST; and

FIGS. 5A-5B are graphs of assays showing the stability of EV71 according to Embodiment 6 of the present invention.

### DETAILED DESCRIPTION OF THE PREFERRED EMBODIMENT

### Incubation of Cells and Viruses

Two cell lines, SK-N-SH and RD cell lines, are used in the present invention, wherein SK-N-SH cell line is Human neuroblastoma cell line, and RD cell line is Human mesenchymal rhabdomyosarcoma cell line. These two cell lines are incubated in DMEM medium supplemented with 10% calf serum, 100 IU/ml penicillin, and 100 mg/ml streptomycin.

In addition, RD cell line infected with EV71 is incubated in DMEM medium supplemented with or without sugars (i.e. monosaccharides). EV71 is incubated in DMEM medium containing sugars in the following assays.

### Embodiment 1

### Binding Assay Between EV71 and Various Monosaccharides

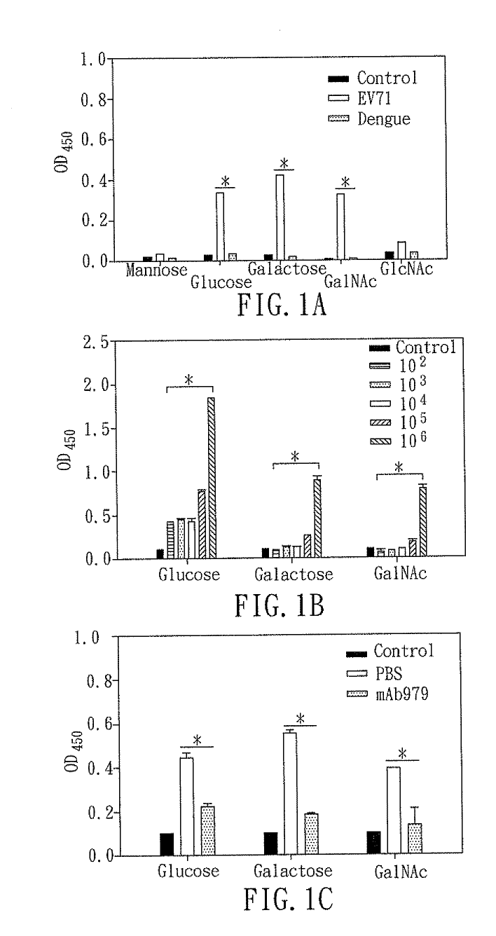

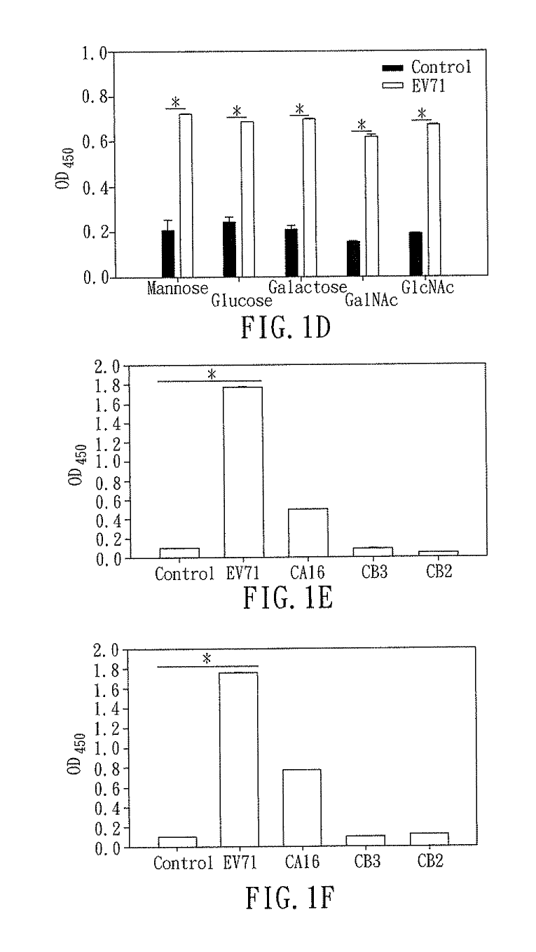

Enzyme-linked immunosorbent assay (ELISA) was used to detect the binding activities between EV71 and monosaccharides in the present embodiment. First, EV71 was added into a 96-well plate (Genesis, Taiwan) and bound to anti-EV71 antibody coated on the 96-well plate. Then, biotin-labeled monosaccharide polymers, such as glucose-PAA (polyacrylamide), mannose-PAA, galactose-PAA, N-acetyl-galactosamine-PAA (GalNAc-PAA), and N-acetyl-glucosamine-PAA (GlcNAc-PAA) were added into the 96-well plate, and reacted with EV71 at room temperature. After 2 hours, streptoavidin-HRP (R&D System, Minneapolis, Minn.) was added into the 96-well plate, and the absorption of streptoavidin-HRP was measured with Enzyme immunoassay under OD₄₅₀. The results are shown in FIGS. 1A-1C.

As shown in FIG. 1A, 10⁶ PFU of EV71 can bind monosaccharides of glucose, galactose, and N-acetyl-galactosamine, compared to the control (without any viruses) or 10⁶ PFU of Dengue viruses.

In addition, as shown in FIG. 1B, when the assay was performed with different amounts of EV71 (10 fold serial dilution from 106 PFU to 10 2 PFU), it can be found that the binding activities between EV71 and monosaccharides such as glucose, galactose, and N-acetyl-galactosamine were enhanced as the amount of EV71 was increased. Even though the amount of EV71 was low (10² PFU), the binding activities between EV71 and glucose can be significantly observed. Herein, control, as showed in FIG. 1B means the absorption of streptoavidin-BRP in the group without any viruses being added.

Furthermore, as shown in FIG. 1C, when biotin labeled glucose, galactose and N-acetyl-galactosamine were dissolved in PBS buffer or diluted in 1:1000 diluted anti-EV71 IgG (mAb979) containing PBS buffer, it can be found that the binding between EV71 and monosaccharides can further be inhibited by the anti-EV71 IgG (mAb979). These results show that there is specific binding between EV71 and monosaccharides. Herein, control, as showed in FIG. 1C means the absorption of streptoavidin-HRP in the group without any viruses being added.

### Embodiment 2

### Binding Assay Between EV71 and Various Monosaccharides

ELISA was also performed to detect the binding activities between EV71 and monosaccharides in the present embodiment, and the process of ELISA of the present embodiment is similar to that of Embodiment 1. First, 10⁶ PFU of EV71 was added into a 96-well plate coated with glucose-PAA, mannose-PAA, galactose-PAA, N-acetyl-galactosamine-PAA, and N-acetyl-glucosamine-PAA. Then, anti-EV71 antibody and HRP-conjugated goat anti-mouse IgG antibody were sequentially added into the 96-well plate. The absorption of HRP was measured with Enzyme immunoassay under OD₄₅₀, and the results are shown in FIG. 1D.

As shown in FIG. 1D, glucose, mannose, galactose, N-acetyl-galactosamine, and N-acetyl-glucosamine can specifically bind to EV71, but no specific binding was observed in the control group without adding EV71.

In addition, specific bindings between different enteroviruses and monosaccharides were also detected. First, 10⁶ PFU of enteroviruses, EV71, Coxsackievirus A16 (CA16), Coxsackievirus B3 (Cox B3, CB3), and Coxsackievirus B2 (Cox B2, CB2), were added in to a 96-well plate coated with glucose-PAA and galactose-PAA. Then, anti-EV71, CA16, CB3 and CB2 antibodies, and HRP-conjugated goat anti-mouse IgG antibodies were sequentially added into the 96-well plate, and the absorption of HRP was measured with Enzyme immunoassay under OD₄₅₀. The results are shown in FIGS. 1E and 1F, wherein control, as showed in the figures means the absorption of streptoavidin-HRP in the group without any viruses being added.

FIG. 1E shows that glucose can specifically bind to Enterovirus species A viruses, such as EV71 and CA 16, and FIG. 1F shows that galactose also can specifically bind to Enterovirus species A virus. In addition, high binding activity between EV71 and glucose or galactose was observed, as shown in FIGS. 1E and 1F. However, other enteroviruses such as CB2 and CB3 do not show any binding activity to glucose or galactose.

According to the results of Embodiments 1 and 2, and the results shown in FIGS. 1A-1F, Enterovirus species A viruses can bind to glucose, galactose, or N-acetyl-galactosamine, and the binding between EV71 and monosaccharides is especially high.

### Embodiment 3

### Binding Assay Between EV71 and Various Lectins

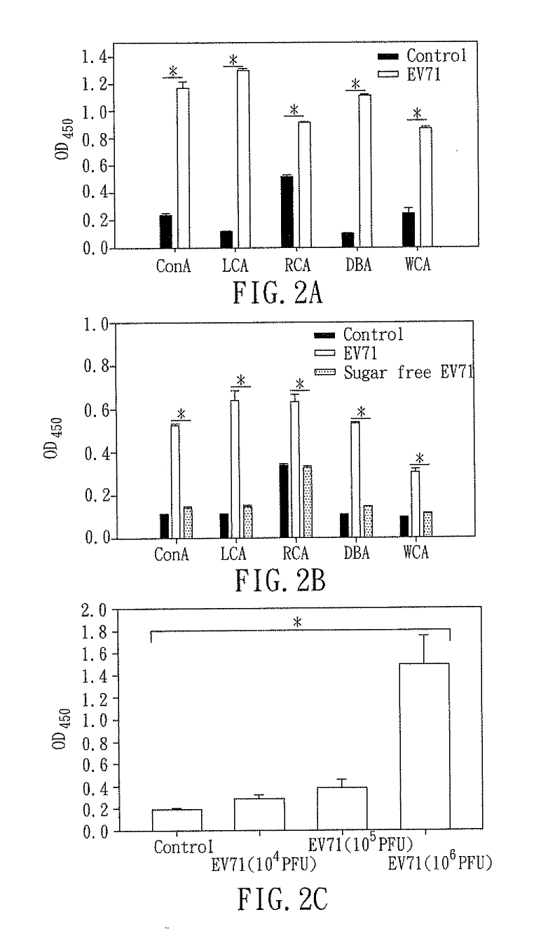

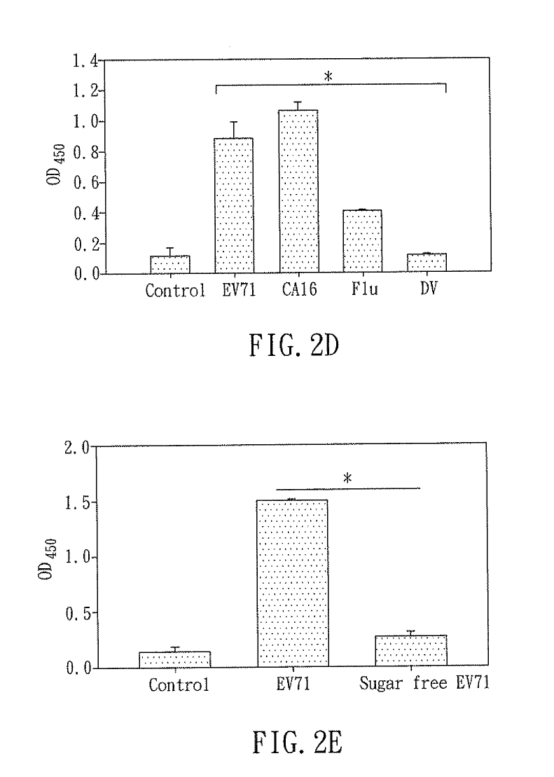

ELISA was used to detect the binding activities between EV71 and lectins in the present embodiment. First, 10⁶ PFU of EV71 was added into a 96-well plate coated with Con A, LCA, WGA, DBA, and RCA. Then, anti-EV71 antibody and HRP-conjugated goat anti-mouse IgG antibody were sequentially added into the 96-well plate. The absorption of HRP was measured with Enzyme immunoassay under OD₄₅₀, and the results are shown in FIG. 2A. Herein, control, as showed in FIG. 2A means the absorption of HRP in the group without any viruses being added.

In addition, 10⁶ PFU of EV71 incubated in glucose-contained or glucose-free medium was added in to a 96-well plate coated with Con A, LCA, WGA, DBA, and RCA. Then, anti-EV71 antibody and HRP-conjugated goat anti-mouse IgG antibody were sequentially added into the 96-well plate. The absorption of HRP was measured with Enzyme immunoassay under OD₄₅©, and the results are shown in FIG. 2B. Herein, control, as showed in FIG. 2B means the absorption of HRP in the group without any viruses being added. The results show that EV71 incubated in sugar-free medium cannot bind to lectins. It means that the monosaccharides such as glucose may first bind to EV71 during the formation of EV71 viral particles, and the monosaccharides bound on EV71 may further participate in the binding between EV71 and lectins. Hence, the binding between EV71 and lectins is accomplished through monosaccharides.

Except the aforementioned lectins, the binding activity between EV71 and mammalian lectin such as galectin-1 was also detected in the present embodiment. First, a different amount of EV71 (10 fold serial dilution from 10⁶ PFU to 10⁴ PFU) was incubated in a 96-well plate coated with galectin-1. Then, anti-EV71 antibody and HRP-conjugated goat anti-mouse IgG antibody were sequentially added into the 96-well plate. The absorption of HRP was measured with Enzyme immunoassay under OD₄₅₀, and the results are shown in FIG. 2C. The results show that EV71 binds to galactin-1, and the amount of bound EV71 is increased as the amount of EV71 added is raised. Herein, control, as showed in FIG. 2C means the absorption of HRP in the group without any viruses being added.

In addition, 106 PFU of different viruses including EV71, CA16, influenza virus (Flu) or dengue virus (DV) were added into a 96-well plate coated with galactin-1. Then, anti-EV71 antibody and HRP-conjugated goat anti-mouse IgG antibody were sequentially added into the 96-well plate. The absorption of HRP was measured with Enzyme immunoassay under OD₄₅₀, and the results are shown in FIG. 2D. The result shows that galactin-1 only specifically binds to Enterovirus species A viruses (EV71 and CA 16), but does not bind to influenza virus and dengue virus. Herein, control, as showed in FIG. 2D means the absorption of HRP in the group without any viruses being added.

Furthermore, 10⁶ PFU of EV71 incubated in glucose-contained or glucose-free medium was added in to a 96-well plate coated with galactin-1. Then, anti-EV71 antibody and HRP-conjugated goat anti-mouse IgG antibody were sequentially added into the 96-well plate. The absorption of HRP was measured with Enzyme immunoassay under OD₄₅₀, and the results are shown in FIG. 2E. The results show that EV71 incubated in sugar-free medium cannot bind to galactin-1. It means that the monosaccharides such as glucose may first bind to EV71 during the formation of EV71 viral particles, and the monosaccharides bound on EV71 may further participate in the binding between EV71 and galactin-1. This result consists with the result shown in FIG. 2B.

According to the results of Embodiment 3, and the results shown in FIGS. 2A-2E, Enterovirus species A viruses can bind to lectins including galactin-1 through monosaccharides. Hence, when a sample is screened for the presence of enteroviruses, lectins and monosaccharides can be used together to improve the effect of enterovirus screening.

### Embodiment 4

### Assay for Detecting the Competition Between Monosaccharides and EV71 or Lectins

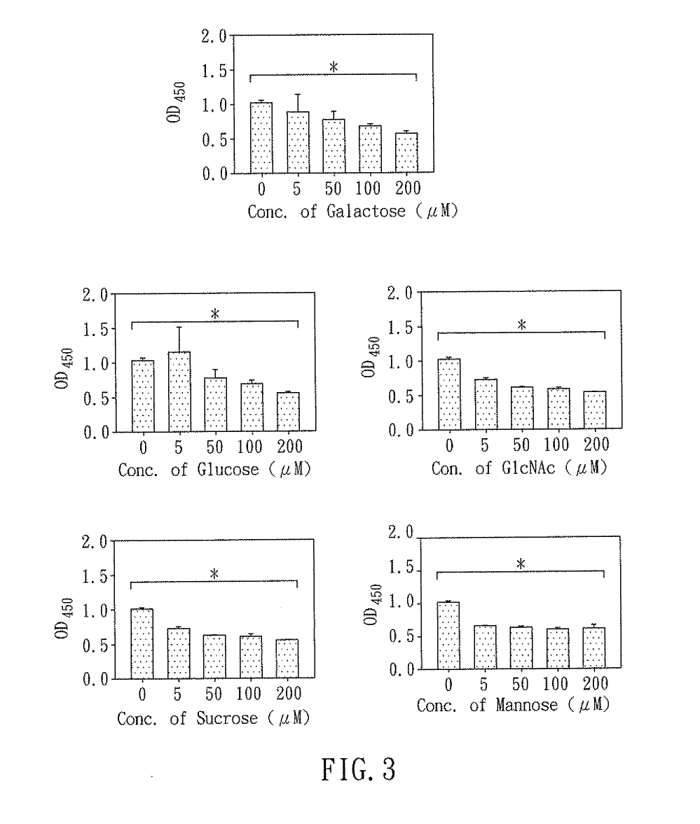

ELISA was used to detect the competition between monosaccharides and EV71 or lectins. First, EV71 was incubated in a medium supplemented with galactose, glucose, N-acetyl galactosamines, sucrose, or mannose with different concentration (conc.) at 4° C. for 2 hours. The incubated EV71 was added into a 96-well plate coated with gelectin-1, and then anti-EV71 antibody and HRP-conjugated goat anti-mouse IgG antibody was added into the 96-well plate. The absorption of HRP was measured with Enzyme immunoassay under OD₄₅₀, and the results are shown in FIG. 3. As shown in FIG. 3, when EV71 was incubated with medium glucose, galactose, or N-acetyl galactosamines, the binding between EV71 and lectins was partially inhibited through the competition of the monosaccharides. It is because the monosaccharides bound on the EV71 may first bind to lectins, so the binding between lectins and EV71 may further be inhibited.

Hence, when enteroviruses are purified with monosaccharides, monosaccharides or lectins can first be coated on a carrier such as a 96-well plate, and then an enterovirus-containing solution is mixed with the carrier. Next, a highly concentrated monosaccharide solution is added, and the monosaccharides contained in the monosaccharide solution can compete with the monosaccharides or lectins coated on the carrier to separate the enterovirus from the carrier.

### Embodiment 5

### Enhancement of EV71 Replication by Use of Monosaccharides

Plaque assay was used to understand the relation between the monosaccharides and the replication of EV71 in the present embodiment.

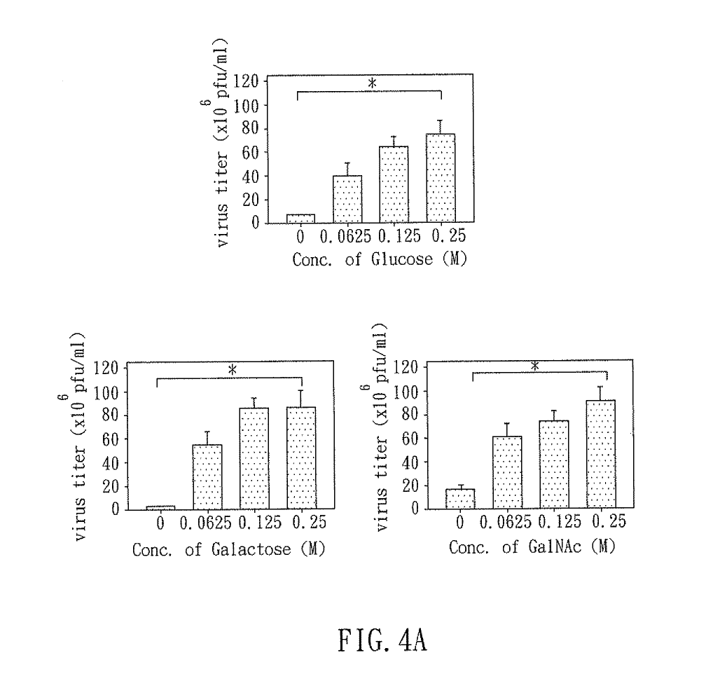

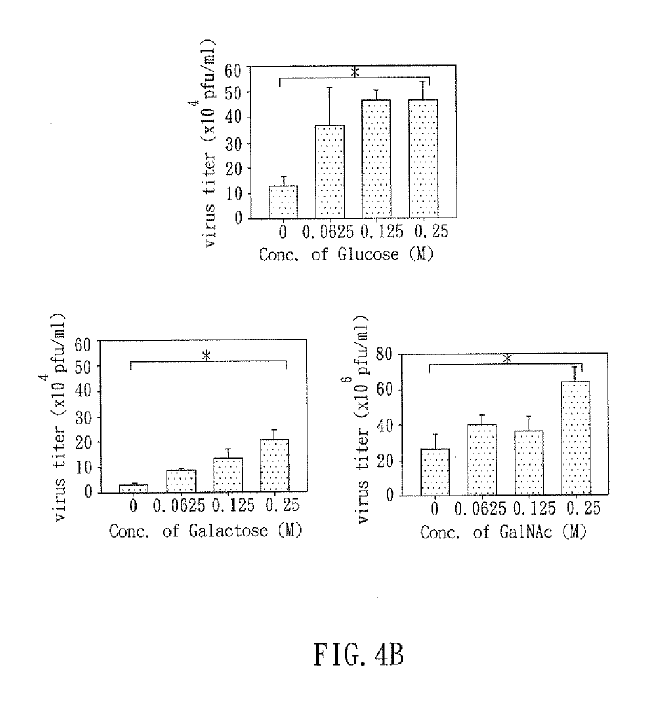

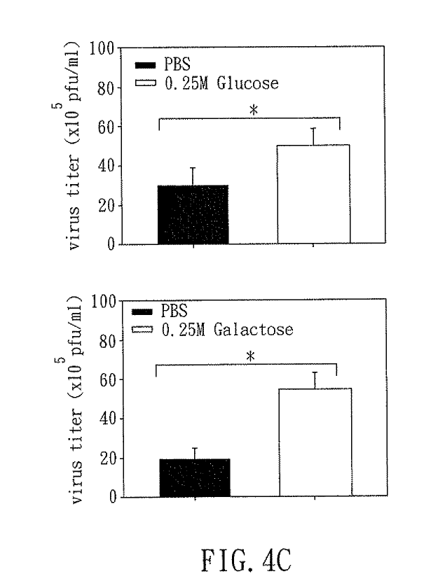

Host cells, SK-N-SH cells (2×10⁵ cells/well), were seeded in a 24-well plate, and incubated for 16-18 hours to form a monolayer cell. SK-N-SH cells were infected with EV71, which was incubated with different concentrations of glucose, galactose or N-acetylgalactosamine (0.625 M, 0.125M, and 0.25M). After 1 hour incubation at 37° C., DMEM with 1.6% methylcellulose and 2% FBS was added to incubate at 37° C. for 72 hours. Crystal violate was overlaid to determine plaque formation, and the quantitative results are shown in FIG. 4A, wherein the longitudinal axis shows the virus titer. As shown in FIG. 4A, glucose, galactose, and N-acetylgalactosamine can all enhance the production of EV71 on SK-N-SH cells.

The following assays are performed to understand that monosaccharides facilitate virus replication at a stage of virus absorption onto host cells, or at a stage after the virus infected host cells.

First, SK-N-SH cells were infected with EV71, which were incubated with different concentrations of glucose, galactose or N-acetylgalactosamine (0.625 M, 0.125M, and 0.25M). After 1 hour incubation at 37° C., unbound viruses were washed away by PBS, DMEM with 1.6% methylcellulose and 2% FBS was added to incubate at 37° C. for 72 hours. Crystal violate was overlaid to determine plaque formation, and the quantitative results are shown in FIG. 4B. As shown in FIG. 4B, glucose, galactose, and N-acetylgalactosamine can all enhance the production of EV71 on SK-N-SH cells. This result indicates that monosaccharides can enhance the absorption of EV71 onto host cells, so the replication of EV71 can further be enhanced.

In addition, SK-N-SH cells were infected with EV71 in a glucose-containing medium. After 1 hour incubation at 37° C., viruses, which were unbound on the 24-well plate were washed away with PBS. Then, the infected host cells were incubated in a medium containing 0.25M glucose or galactose (supplemented with 1.6% methylcellulose and 2% FBS) at 37° C. for 72 hours. Crystal violate was overlaid to determine plaque formation, and the quantitative results are shown in FIG. 4C. As shown in FIG. 4C, monosaccharides can facilitate the absorption of EV71 onto host cells to increase the amount of infected host cells, and also the replication of EV71 after the virus infected the host cell.

According to the aforementioned results, monosaccharides facilitate not only virus absorption, but also virus replication. Hence, host cells can be incubated in a medium supplemented with monosaccharides at a stage of virus absorption or after virus infection, in order to produce enteroviruses in a large scale.

### Embodiment 6

### Enhancement of EV71 Stability by Use of Monosaccharides

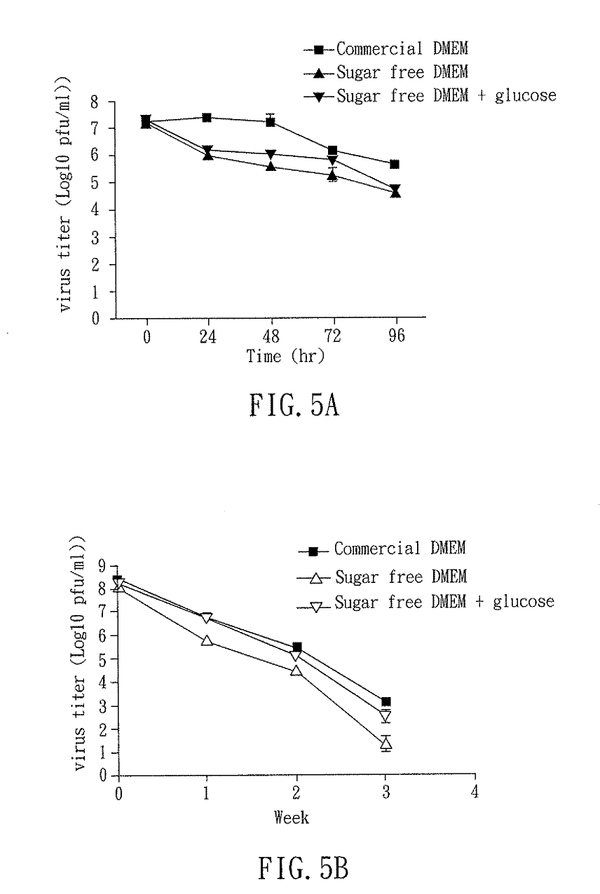

The same amount of EV71 in DMEM, sugar free DMEM, or sugar free DMEM with addition of glucose were incubated at 37° C., and then the stability of EV71 was detected with plaque assay. As shown in FIGS. 5A and 5B, the stability of EV71 incubated in DEME containing glucose is better than that incubated in sugar free DMEM with addition of glucose, and much better than that incubated in sugar free DMEM. These results indicate that glucose can enhance the stability of EV71.

### Embodiment 7

### Production of Vaccines Against EV71

The host cells infected with EV71 were incubated in a glucose-containing medium, and then the host cells were lysed to obtain an EV71-containing solution. The EV71-containing solution was centrifuged, the pellets were removed, and the supernatant was mixed with a buffer containing 42% PEG8000 and 6% NaCl and incubated at 4° C. overnight. After centrifugation, the supernatant was removed, and the pellets were re-suspended with TES buffer. After further centrifugation, the supernatant was removed, and the pellets were extracted with TES buffer many times to obtain an EV71-containing solution. Then, the EV71-containing solution was mixed with carriers coated with glucose, and EV71 was purified with a glucose gradient. EV71 can be separated from the carriers through the competition of glucose between the glucose gradient and the carriers. The obtained EV71 solution was dialyzed with PBS, and finally the purified EV71 was suspended in PBS.

The purified EV71 was added into 0.1 v/v % formaldehyde (37%), and incubated at 37° C. for 2 hours to deactivate EV71. The deactivated EV71 was mixed with alum hydroxide with a final concentration of 660 μg/ml, and incubated for 30 mins to obtain a vaccine against EV71.

Although the present invention has been explained in relation to its preferred embodiment, it is to be understood that many other possible modifications and variations can be made without departing from the spirit and scope of the invention as hereinafter claimed.

What is claimed is:

1. A method for screening a sample for the presence of an enterovirus, comprising the following steps:
(A) providing a sample, and a carrier, wherein monosaccharides are bound to a surface of the carrier, and the monosaccharides have a binding affinity to the enterovirus;
(B) contacting the sample with the carrier;
(C) removing components of the same that does not bind to the monosaccharides on the carrier;
(D) providing a detection unit, and contacting the detection unit with the carrier, wherein the detection unit binds to the sample bound to the monosaccharides on the carrier; and
(E) measuring a signal of the detection unit, wherein when the signal of the detection unit is detected, it represents that the enterovirus exists in the sample.

2. The method as claimed in claim 1, wherein the enterovirus is Enterovirus species A virus.

3. The method as claimed in claim 2, wherein the Enterovirus species A virus is Enterovirus 71, or Coxsackievirus A16.

4. The method as claimed in claim 1, wherein the monosaccharides are glucoses, galactoses, or N-acetyl galactosamines.

5. The method as claimed in claim 1, wherein the monosaccharides are directly bound to the surface of the carrier; or the monosaccharides are bound to the surface of the carrier through lectins, in the step (A).

6. The method as claimed in claim 5, wherein the lectins are galectin-1, Concanavalin A, Lens culinaris agglutinin, Wheat germ agglutinin, Dolichos biflorus, or Ricinus lectin.

7. The method as claimed in claim 1, wherein the detection unit comprises: an anti-enterovirus antibody.

8. The method as claimed in claim 7, wherein the detection unit further comprises: a horseradish peroxidase-conjugated antibody, which connects to the anti-enterovirus antibody.

9. A method for purifying an enterovirus, comprising the following steps:
(A) providing carriers, wherein monosaccharides are bound to surfaces of the carriers;
(B) mixing an enterovirus-containing solution with the carriers, wherein enteroviruses contained in the enterovirus-containing solution bind to the monosaccharides on the carriers;
(C) washing the carriers to remove components contained in the enterovirus-containing solution, which are not bound to the carriers; and
(D) providing a monosaccharide solution to separate the enteroviruses from the monosaccharides on the carrier.

10. The method as claimed in claim 9, wherein the enterovirus is Enterovirus species A virus.

11. The method as claimed in claim 10, wherein the Enterovirus species A virus is Enterovirus 71, or Coxsackievirus A16.

12. The method as claimed in claim 9, wherein the monosaccharides are glucoses, galactoses, or N-acetyl galactosamines.

13. The method as claimed in claim 9, wherein the monosaccharides are directly bound to the surface of the carrier; or the monosaccharides are bound to the surface of the carrier through lectins, in the step (A).

14. The method as claimed in claim 13, wherein the lectins are galectin-1, Concanavalin A, Lens culinaris agglutinin, Wheat germ agglutinin, Dolichos biflorus, or Ricinus lectin.

15. A method for mass-producing an enterovirus, comprising the following steps:
(A) providing host cells, and an enteroviruses;
(B) mixing the host cells and the enteroviruses in a monosaccharide-containing medium to transfect the enteroviruses into the host cells;
(C) incubating the host cells transfected with the enteroviruses; and
(D) extracting the enteroviruses from the host cells.

16. The method as claimed in claim 15, wherein the host cells transfected with the enteroviruses are incubated in a monosaccharide-containing medium, in the step (C).

17. The method as claimed in claim 15, wherein the enteroviruses are Enterovirus species A virus.

18. The method as claimed in claim 17, wherein the Enterovirus species A virus is Enterovirus 71, or Coxsackievirus A16.

19. The method as claimed in claim 15, wherein monosaccharides contained in the monosaccharide-containing medium are glucoses, galactoses, or N-acetyl galactosamines.

20. The method as claimed in claim 16, wherein monosaccharides contained in the monosaccharide-containing medium are glucoses, galactoses, or N-acetyl galactosamines.

21. A method for manufacturing an enterovirus vaccine, comprising the following steps:
(A) providing host cells and enteroviruses;
(B) mixing the host cells and the enteroviruses in a monosaccharide-containing medium to transfect the enteroviruses into the host cells;
(C) incubating the host cells transfected with the enteroviruses;
(D) extracting the enteroviruses from the host cells; and
(E) deactivating the enteroviruses extracted from the host cells.

22. The method as claimed in claim 21, wherein the enteroviruses are Enterovirus species A virus.

23. The method as claimed in claim 22, wherein the Enterovirus species A virus is Enterovirus 71, or Coxsackievirus A16.

24. The method as claimed in claim 21, wherein monosaccharides contained in the monosaccharide-containing medium are glucoses, galactoses, or N-acetyl galactosamines.

25. The method as claimed in claim 21, wherein the host cells transfected with the enterovirus are incubated in a monosaccharide-containing medium, in the step (C).

26. The method as claimed in claim 21, wherein the enteroviruses extracted from the host cells are deactivated with formaldehyde.

27. The method as claimed in claim 21, wherein the step (D) comprises the following steps:
(D1) providing carriers, wherein monosaccharides are bound on surfaces of the carriers;
(D2) lysing the host cells to obtain an enterovirus-containing solution;
(D3) mixing the enterovirus-containing solution with the carriers, wherein enteroviruses contained in the enterovirus-containing solution bind to the monosaccharides on the carriers;
(D4) washing the carriers to remove components contained in the enterovirus-containing solution, which are not bound to the carriers; and
(D5) providing a monosaccharide solution to separate the enteroviruses from the monosaccharides on the carrier.

28. The method as claimed in claim 27, wherein the monosaccharides are glucoses, galactoses, or N-acetyl galactosamines, in the step (D1).

29. The method as claimed in claim 27, wherein the monosaccharides are directly bound to the surface of the carrier; or the monosaccharides are bound to the surface of the carrier through lectins, in the step (D1).
